(19) United States
(12) Patent Application Publication
(10) Pub. No.: US 2023/0187073 A1
(43) Pub. Date: Jun. 15, 2023

(54) SYSTEMS AND METHODS FOR PREDICTING, DETECTING, AND MONITORING OF ACUTE ILLNESS

(71) Applicant: EVIDATION HEALTH, INC., San Mateo, CA (US)

(72) Inventors: Luca FOSCHINI, Santa Barbara, CA (US); Eamon CADDIGAN, Philadelphia, PA (US); Filip JANKOVIC, Santa Barbara, CA (US); Arinbjörn KOLBEINSSON, Berlin (DE); Benjamin BRADSHAW, San Diego, CA (US); Raghunandan KAINKARYAM, Cincinnati, OH (US)

(21) Appl. No.: 18/148,991

(22) Filed: Dec. 30, 2022

Related U.S. Application Data
(63) Continuation of application No. PCT/US22/81465, filed on Dec. 13, 2022.
(60) Provisional application No. 63/373,671, filed on Aug. 26, 2022.
(60) Provisional application No. 63/289,372, filed on Dec. 14, 2021.

(51) Int. Cl. G16H 50/20; G16H 50/30

(52) U.S. Cl. CPC G16H 50/20; G16H 50/30

(57) ABSTRACT

In an aspect, a computer-implemented method for assembling a pool of high-risk subjects for developing an acute illness is disclosed. The method comprises obtaining, from a plurality of subjects, (i) one or more responses to one or more health queries, and (ii) geographic incidence data for the acute illness. The method next comprises predicting, using a machine learning model, a risk of developing the acute illness for the plurality of subjects based on the one or more responses and the geographic incidence data. The method next comprises identifying the pool of high-risk subjects from the plurality of subjects, wherein the risk of developing the acute illness for each subject of the pool of high-risk subjects satisfies a threshold. Finally, the method comprises outputting the pool of high-risk subjects.

### CROSS-REFERENCE

This application is a continuation of International Application No. PCT/US2022/081465, filed Dec. 13, 2022, which claims the benefit of U.S. Provisional Application No. 63/289,372, filed Dec. 14, 2021, and U.S. Provisional Application No. 63/373,671, filed Aug. 26, 2022, each of which is entirely incorporated herein by reference.

### BACKGROUND

Acute health conditions or illnesses, characterized by rapid onset and recovery, may present challenges for the researchers who track and monitor their prevalences within, and effects on, a population. Researchers may have short time windows in which to study these acute illnesses and may thus need to gather suitable subjects for analysis quickly. Suitable subjects may be those most likely to contract an acute illness or suffer an acute health condition of interest within a short time period.

### SUMMARY

There is a need for a system that can select a suitable group of candidates that may be observed for predicting and/or monitoring of an acute illness or health condition. The systems, the methods, the computer-readable media, and the techniques described herein may provide researchers with high-risk pools of participants that either are likely to contract acute illnesses or who have already contracted them.

In some cases, methods, systems, computer-readable media, and techniques described herein may implement a machine learning model to identify the factors that best predict infection of acute illnesses or acute health conditions (e.g., COVID-19 infection) in a subject. Information determined from this machine learning analysis may assist with predicting, detecting, and monitoring acute illnesses in a subject or in a population. The methods, the systems, the computer-readable media, and the techniques described herein may analyze factors such as local prevalence of the acute illness, based on, for example, information from the Centers for Disease Control and Prevention (CDC). Various other factors may be used to predict infection of a subject, a zone, or a population for the acute illness including the number of potentially risky contacts (e.g., household size and residential situation), location (e.g., living in a city with numerous COVID-19 cases at the time of recruitment), and working in a risky occupation (e.g., healthcare jobs). In some cases, answers to a survey with health queries or wearable device data may also be ingested by the machine learning model to predict infection risk. Based on some or all these factors, the machine learning model may then calculate an infection risk score for a subject, a zone, or a population.

Research studies developing screening tools, diagnostics, and preventative treatments may be improved by observing a large number of transitions from a healthy state to a disease state over a short observation period. By implementing the methods, the systems, the computer-readable media, and the techniques described herein, the ability to predict beforehand which individuals will get sick over a short observation period may be improved; thereby enabling increased observation of more of these transitions faster or with fewer participants in the study. A group of subjects selected using the methods herein may be used in a study to detect onset of acute illnesses from wearable sensor data, which enables observation from acute illness-negative to acute illness-positive over the study. A specific example of preventative treatment development is studies to validate vaccine efficacy. In such studies, a certain number of infections needs to be observed in order to discern a statistically significant effect of the vaccine arm vs. the placebo arm. By enriching for incidence of illness (e.g., increasing the number of transitions from negative to positive) fewer participants will need to be recruited for the same number of infectious events observed, thus reducing time and resources needed by the study.

In some cases, a health reward platform is provided that encourages its members to develop healthy habits such as walking, meditating, and logging meals. The health reward platform may also incentivize members to participate in research by completing surveys and sharing data from commercially available wearable devices provided by companies, such as Fitbit®. As one example, the health reward platform may be used to monitor annual waves of infectious diseases (e.g., influenza, COVID-19, etc.). With the platform, recruitment for acute illness studies or trials may be done very quickly and in a decentralized manner. For example, participants may be able to participate in the study or trial without having to travel to a center. Rather, testing kits may be mailed and surveys can be taken frequently via a mobile device. Accordingly, these capabilities may improve rapid response to crises, such as COVID-19, and speed up enrollment of the participants for the study or trial.

In some cases, subjects with the highest risk scores may be targeted for recruitment for studies or trials (and in some cases, the subjects may be balanced for age, sex, and ethnicity). In some cases, the subjects with the highest risk score may in addition or in alternative, be alerted to their heightened risk for the acute illness so that they may seek appropriate medical care or precautions. For example, the subjects with the highest risk score may be prioritized for vaccine distribution, can be flagged for preventative mandates (e.g., quarantines, masks, school or business closures, public health campaigns or education, etc.), or can be notified (e.g., via notification transmission via SMS or emergency communication channels) in advance of potential outbreaks or contractions of the acute illness.

In one real-world example, the machine learning model was applied to recruiting subjects who are currently negative for COVID-19, but are at risk for catching COVID-19. In this real-world example, applying the systems, the methods, the computer-readable media, and the techniques described herein, the machine learning model was used to select subjects with incidence rates of COVID-19 of a factor of 4 to 7 times higher than the actual incidence rate seen in the placebo arm for three vaccine trials run in the US, which can be considered as a baseline for incidence rate in a representative, non-enriched, population. Advantageously, the machine learning model may enable trials or studies (e.g., for developing vaccines) to cut their candidate populations by the same factor (four to seven times) or allow studies or trials to be performed much more quickly, thereby reducing costs, saving resources, and potentially improving public health and safety.

In some cases, the systems, the methods, the computer-readable media, and the techniques described herein perform operations including: obtaining, from a plurality of subjects, (i) one or more responses to one or more health queries, and (ii) geographic incidence data for the acute illness; predicting, using a machine learning model, a risk of developing the acute illness for the plurality of subjects based on the one or more responses and the geographic incidence data; identifying the pool of high-risk subjects from the plurality of subjects, wherein the risk of developing the acute illness for each subject of the pool of high-risk subjects satisfies a threshold; and outputting the pool of high-risk subjects.

In some cases, the systems, the methods, the computer-readable media, and the techniques described herein perform operations including: obtaining wearable device sensor data from the subject; obtaining, via a mobile device, a response to a health query of whether the subject has a symptom associated with the acute illness; predicting, using a machine learning model, a risk of the subject developing the acute illness based at least in part on the wearable device sensor data and the response to the health query; and causing an electronic display to indicate the risk to the subject.

### BRIEF DESCRIPTION OF THE DRAWINGS

A better understanding of the features and advantages of the present subject matter will be obtained by reference to the following detailed description that sets forth illustrative embodiments and the accompanying drawings of which:

FIG. 1 shows a non-limiting example of a block diagram of an environment for modeling an outbreak;

FIG. 2A shows a non-limiting example of a flowchart illustrating a method for assembling a pool of high-risk subjects for developing an acute illness;

FIG. 2B shows a non-limiting example of a flowchart illustrating a method for presenting information to a subject about an acute illness;

FIG. 3 shows a non-limiting example of data illustrating feature importance;

FIG. 4 shows a non-limiting example of a workflow for collecting data;

FIG. 5A shows a non-limiting example of a workflow for onboarding and enrolling users;

FIG. 5B shows a non-limiting example of a workflow for user participation;

FIGS. 6A-6F show various a non-limiting examples of user interfaces used for collecting data;

FIG. 7 shows a non-limiting example of a computing device; in this case, a device with one or more processors, memory, storage, and a network interface; and

FIG. 8 shows a non-limiting example of a random forest with a plurality of decision trees.

### DETAILED DESCRIPTION

Recruiting for clinical trials may be conducted by techniques such as advertisements, direct provider contact, etc. However, the techniques of advertising, direct provider contact, etc. may be costly (in terms of financial resources, time, communication resources, etc.). As such, the techniques of advertising, direct provider contact, etc. may be too slow to achieve enrollment for spreading and contracting of acute illnesses. The slowness of the techniques of advertising, direct provider contact, etc. may be particularly problematic for infectious diseases, such as COVID-19.

The methods, the systems, the computer-readable media, and the techniques described herein may predict, detect, or monitor subjects, zones, populations, etc. that are at high risk for an acute illness (e.g., infectious disease). These high-risk subjects, zones, populations, etc. can be prioritized for vaccine distribution, can be flagged for preventative mandates (e.g., quarantines, masks, school or business closures, public health campaigns or education, etc.), or can be notified (e.g., via notification transmission via SMS or emergency communication channels) in advance of potential outbreaks or contractions of the acute illness. In some cases, the methods, the systems, the computer-readable media, and the techniques can be used to recruit subjects, zones, populations, etc. for participation in studies or trials, based on, for example, their risk for the acute illness.

### Certain Definitions

Unless otherwise defined, all technical terms used herein have the same meaning as commonly understood by one of ordinary skill in the art to which the present subject matter belongs.

As used in this specification and the appended claims, the terms “artificial intelligence,” “artificial intelligence techniques,” “artificial intelligence operation,” and “artificial intelligence algorithm” generally refer to any system or computational procedure that may take one or more actions to enhance or maximize a chance of achieving a goal. The term “artificial intelligence” may include “generative modeling,” “machine learning” (ML), or “reinforcement learning” (RL).

As used in this specification and the appended claims, the terms “machine learning,” “machine learning techniques,” “machine learning operation,” and “machine learning model” generally refer to any system or analytical or statistical procedure that may progressively improve computer performance of a task.

As used in this specification and the appended claims, “acute illness” may generally refer to an abnormal or detrimental condition of the body that may develop rapidly and last for a short period, including infectious diseases. The term “acute illness” may be used interchangeably herein with “acute condition” or “acute health condition.”

As used in this specification and the appended claims, “some embodiments,” “further embodiments,” or “a particular embodiment,” means that a particular feature, structure, or characteristic described in connection with the embodiment is included in at least one embodiment. Thus, the appearances of the phrase “in some embodiments,” or “in further embodiments,” or “in a particular embodiment” in various places throughout this specification are not necessarily all referring to the same embodiment. Furthermore, the particular features, structures, or characteristics may be combined in any suitable manner in one or more embodiments.

As used in this specification and the appended claims, when the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

As used in this specification and the appended claims, when the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used in this specification, “or” is intended to mean an “inclusive or” or what is also known as a “logical OR,” wherein when used as a logic statement, the expression “A or B” is true if either A or B is true, or if both A and B are true, and when used as a list of elements, the expression “A, B or C” is intended to include all combinations of the elements recited in the expression, for example, any of the elements selected from the group consisting of A, B, C, (A, B), (A, C), (B, C), and (A, B, C); and so on if additional elements are listed. As such, any reference to “or” herein is intended to encompass “and/or” unless otherwise stated.

As used in this specification and the appended claims, the indefinite articles “a” or “an,” and the corresponding associated definite articles “the” or “said,” are each intended to mean one or more unless otherwise stated, implied, or physically impossible. Yet further, it should be understood that the expressions “at least one of A and B, etc.,” “at least one of A or B, etc.,” “selected from A and B, etc.” and “selected from A or B, etc.” are each intended to mean either any recited element individually or any combination of two or more elements, for example, any of the elements from the group consisting of “A,” “B,” and “A AND B together,” etc.

As used in this specification and the appended claims “about” or “approximately” may mean within an acceptable error range for the value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where values are described in the application and claims, unless otherwise stated the term “about” meaning within an acceptable error range for the particular value may be assumed.

### Examples of Machine Learning Methodologies

The systems, the methods, the computer-readable media, and the techniques described herein may use machine learning. In some cases, machine learning may generally involve identifying and recognizing patterns in existing data in order to facilitate making predictions for subsequent data. Machine learning may include a machine learning model (which may include, for example, a machine learning algorithm). Machine learning, whether analytical or statistical in nature, may provide deductive or abductive inference based on real or simulated data.

The machine learning model may be a trained model. Machine learning (ML) may comprise one or more supervised, semi-supervised, self-supervised, or unsupervised machine learning techniques. For example, an ML model may be a trained model that is trained through supervised learning (e.g., various parameters are determined as weights or scaling factors).

Training the machine learning model may include, in some cases, selecting one or more untrained data models to train using a training data set. The selected untrained data models may include any type of untrained machine learning models for supervised, semi-supervised, self-supervised, or unsupervised machine learning. The selected untrained data models be specified based upon input (e.g., user input) specifying relevant parameters to use as predicted variables or other variables to use as potential explanatory variables. For example, the selected untrained data models may be specified to generate an output (e.g., a prediction) based upon the input. Conditions for training the machine learning model from the selected untrained data models may likewise be selected, such as limits on the machine learning model complexity or limits on the machine learning model refinement past a certain point. The machine learning model may be trained (e.g., via a computer system such as a server) using the training data set. In some cases, a first subset of the training data set may be selected to train the machine learning model. The selected untrained data models may then be trained on the first subset of training data set using appropriate machine learning techniques, based upon the type of machine learning model selected and any conditions specified for training the machine learning model. In some cases, due to the processing power requirements of training the machine learning model, the selected untrained data models may be trained using additional computing resources (e.g., cloud computing resources). Such training may continue, in some cases, until at least one aspect of the machine learning model is validated and meets selection criteria to be used as a predictive model.

In some cases, one or more aspects of the machine learning model may be validated using a second subset of the training data set (e.g., distinct from the first subset of the training data set) to determine accuracy and robustness of the machine learning model. Such validation may include applying the machine learning model to the second subset of the training data set to make predictions derived from the second subset of the training data. The machine learning model may then be evaluated to determine whether performance is sufficient based upon the derived predictions. The sufficiency criteria applied to the machine learning model may vary depending upon the size of the training data set available for training, the performance of previous iterations of trained models, or user-specified performance requirements. If the machine learning model does not achieve sufficient performance, additional training may be performed. Additional training may include refinement of the machine learning model or retraining on a different first subset of the training dataset, after which the new machine learning model may again be validated and assessed. When the machine learning model has achieved sufficient performance, in some cases, the machine learning may be stored for present or future use. The machine learning model may be stored as sets of parameter values or weights for analysis of further input (e.g., further relevant parameters to use as further predicted variables, further explanatory variables, further user interaction data, etc.), which may also include analysis logic or indications of model validity in some instances. In some cases, a plurality of machine learning models may be stored for generating predictions under different sets of input data conditions. In some embodiments, the machine learning model may be stored in a database (e.g., associated with server).

ML may comprise one or more of regression analysis, regularization, classification, dimensionality reduction, ensemble learning, meta learning, association rule learning, cluster analysis, anomaly detection, deep learning, or ultra-deep learning. ML may comprise, but is not limited to: k-means, k-means clustering, k-nearest neighbors, learning vector quantization, linear regression, non-linear regression, least squares regression, partial least squares regression, logistic regression, stepwise regression, multivariate adaptive regression splines, ridge regression, principal component regression, least absolute shrinkage and selection operation, least angle regression, canonical correlation analysis, factor analysis, independent component analysis, linear discriminant analysis, multidimensional scaling, non-negative matrix factorization, principal components analysis, principal coordinates analysis, projection pursuit, Sammon mapping, t-distributed stochastic neighbor embedding, AdaBoosting, boosting, gradient boosting, bootstrap aggregation, ensemble averaging, decision trees, conditional decision trees, boosted decision trees, gradient boosted decision trees, random forests, stacked generalization, Bayesian networks, Bayesian belief networks, naïve Bayes, Gaussian naïve Bayes, multinomial naïve Bayes, hidden Markov models, hierarchical hidden Markov models, support vector machines, encoders, decoders, auto-encoders, stacked auto-encoders, perceptrons, multi-layer perceptrons, artificial neural networks, feedforward neural networks, convolutional neural networks, recurrent neural networks, long short-term memory, deep belief networks, deep Boltzmann machines, deep convolutional neural networks, deep recurrent neural networks, or generative adversarial networks.

As described above, the machine learning model may implement a decision tree. A decision tree may be a supervised machine learning algorithm that can be applied to both regression and classification problems. Decision trees may mimic the decision-making process of a human brain. For example, a decision tree may grow from a root (base condition), and when it meets a condition (internal node/feature), it may split into multiple branches. The end of the branch that does not split anymore may be an outcome (leaf). A decision tree can be generated using a training data set according to the following operations: (1) Starting from a root node (the entire dataset), the algorithm may split the dataset in two branches using a decision rule or branching criterion; (2) each of these two branches may generate a new child node; (3) for each new child node, the branching process may be repeated until the dataset cannot be split any further; (4) each branching criterion may be chosen to maximize information gain (e.g., a quantification of how much a branching criterion reduces a quantification of how mixed the labels are in the children nodes). The labels may be the data or the classification that is predicted by the decision tree.

A random forest regression is an extension of the decision tree model that tends to yield more robust predictions by stretching the use of the training data partition. Whereas a decision tree may make a single pass through the data, a random forest regression may bootstrap 50% of the data (e.g., with replacement) and build many trees. Rather than using all explanatory variables as candidates for splitting, a random subset of candidate variables may be used for splitting, which may enable trees that have completely different data and different variables (hence the term random). The predictions from the trees, collectively referred to as the “forest,” may be then averaged together to produce the final prediction. Many trees (e.g., one hundred trees) may be included in a random forest model, with a number (e.g., 3, 6, 10, etc.) of terms sampled per split, a minimum of number (e.g., 1, 2, 4, 10, etc.) of splits per tree, and a minimum split size (e.g., 16, 32, 64, 128, 256, etc.). Random forests may be trained in a similar way as decision trees. Specifically, training a random forest may include the following operations: (1) select randomly k features from the total number of features; (2) create a decision tree from these k features using the same operations as for generating a decision tree; and (3) repeat the previous two operations until a target number of trees is created.

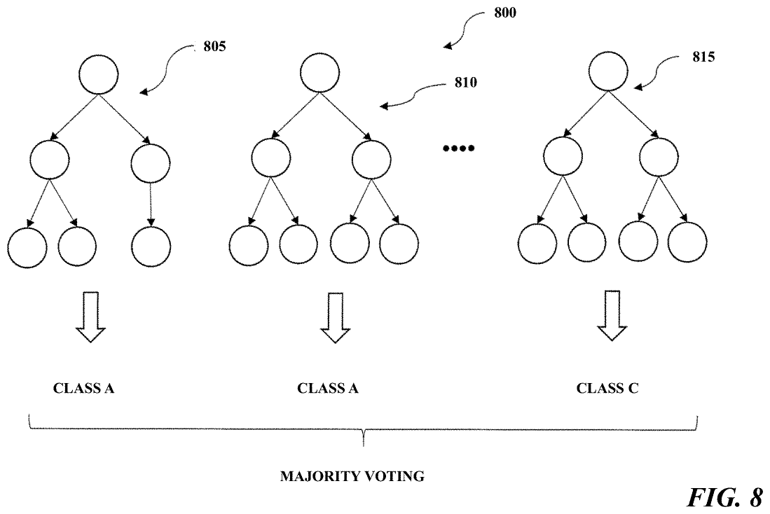

FIG. 8 illustrates a random forest 800. The random forest 800 (which may also be referred to as random forest model) is an ensemble of decision trees 805, 810, and 815 with randomly selected features in each of the decision trees 805, 810, and 815 so that it can provide more stable and accurate outcomes. Outcomes may be determined by majority voting in the case of a classification problem. In the example of FIG. 8, the random forest 800, which has been trained previously by a training method, is used to decide between classifications A, B and C. For example, the random forest 800, with only the three decision trees shown in FIG. 8, would return the classification A by majority voting.

### Acute Illnesses

The systems and methods described herein may be used to recruit subjects for studies of acute illnesses or acute health conditions. Acute illnesses may be severe. Acute illnesses may last a few days or a few weeks. An acute illness may develop in periods of fractions of a second, seconds, minutes, hours, a few days, or a few weeks. Chronic conditions, or long-developing conditions, may cause acute conditions or illnesses. For example, osteoporosis may result in a broken bone.

An acute illness or acute health condition may be a disease. An acute illness or health condition may be an infectious disease. An acute illness may be a viral or bacterial infection. An acute illness may be a respiratory illness such as the common cold, influenza, a coronavirus disease (e.g., coronavirus disease 2019 (COVID-19) or severe acute respiratory syndrome (SARS)), acute respiratory distress syndrome, pharyngitis, acute bronchitis, or acute asthma. An acute illness may be a non-infectious disease, such as acute leukemia, acute myocardial infarction, acute gastroenteritis, or acute hepatitis.

An acute illness or acute health condition may be an injury. An injury may be caused by an external trauma to the body or by a sudden movement of the body, for example, during exercise or a sport. An injury may be a broken bone, a sprain, a strain, a dislocation, a bruise, a cut, a gash, or a tear.

An acute illness or acute health condition may be a mental condition. Non-limiting examples of acute mental conditions may be depressive episodes, manic episodes, psychotic episodes, panic attacks, acute stress disorder, or post-traumatic stress disorder.

### Example Environment

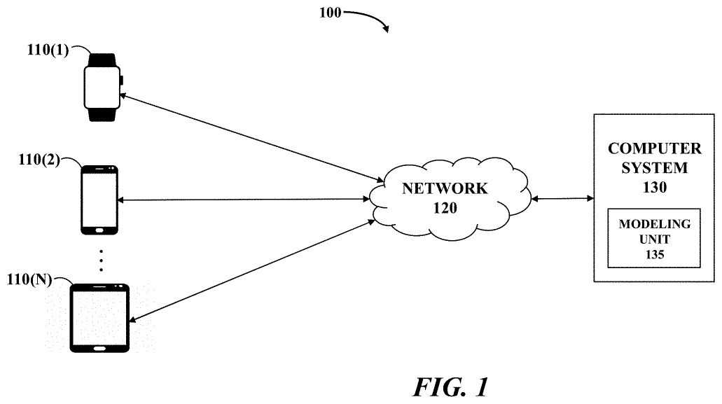

FIG. 1 is a block diagram of an environment 100 for modeling risk of an acute illness to one or more subjects, in accordance with some cases. The environment 100 of FIG. 1 includes one or more user devices 110(1)-110(N), a network 120, and a computer system 130 with a modeling unit 135. At a high level, the user devices 110(1)-110(N) may be used by one or more users (e.g., humans, animals, etc.) to collect or provide, via the network 120, data about the one or more users to the computer system 130 so that the computer system 130, using the modeling unit 135, can predict risk of an acute illness to the one or more subjects or a population (e.g., including at least a portion of the one or more subjects).

The user devices 110(1)-110(N) may be one or more electronic devices. In some cases, the electronic devices may be mobile devices. For example, the mobile devices may be smartphones (e.g., the user device 110(2)) or tablets (e.g., see the user device 110(N)). In some cases, the electronic devices may be wearable devices. For example, the wearable devices may be smartwatches (e.g., the user device 110(1)), smartbands (e.g., a Fitbit® device), smartclothing, smart jewelry, smartshoes, or the like. In some cases, the electronic devices may be a laptop or desktop computer. In some cases, the electronic devices may be gaming devices (e.g., the gaming device may collect data as part of a game or may reward users based on certain collected data or outcomes). In general, the user devices 110(1)-110(N) may be any device suitable for collecting data such as wearable device data, responses to queries, geographical data, demographical data, or any other health data or the like. For example, the user devices 110(1)-110(N) may be any one or more of desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, vehicles, televisions, exercise equipment, video players, digital music players, booklet tablet computers, slate tablet computers, convertible tablet computers, or the like. Each of the user devices 110(1)-110(N) may be linked to, and collect data from, a single user. Each the user devices 110(1)-110(N) may be linked to, and collect data from, multiple users (e.g., a household).

As described, the user devices 110(1)-110(N) can be wearable devices or other device capable of providing physical (e.g., medical, activity, nutrition, etc.) statistics about one or more users. For example, the user devices 110(1)-110(N) can be a dedicated fitness tracker, a pedometer, a sleep tracker, a smart watch, smartphone, or mobile device (e.g., a tablet computer or a personal digital assistant (PDA)) with physical statistic monitoring functionality. For example, the user devices 110(1)-110(N) can be a smartphone of one or more users with an installed physical statistic monitoring application using one or more sensors of the smartphone to measure steps, activity, movement, sleep time, or other physical statistics. In some cases, a user may be associated with multiple devices of the user devices 110(1)-110(N) measuring overlapping or distinct physical statistics about the user. For example, a smartphone may measure sleep behavior or sleep efficiency of the user (e.g., based on when the user uses the device), a pedometer may measure step counts of the user, and a smart exercise machine may measure blood pressure of the user. The physical statistic data gathered by the user devices 110(1)-110(N) can be sent to the computer system 130 directly from the user devices 110(1)-110(N), manually uploaded to the computer system 130 by the associated users or transmitted via a third-party system to the computer system 130. For example, a user may authorize a third-party service associated with the user devices 110(1)-110(N) to transmit physical activity data to the computer system 130. In some cases, a user can interact with the computer system 130 via the user devices 110(1)-110(N). For example, a user may be able to update information about themself stored in the computer system 130 via the user devices 110(1)-110(N). In some cases, a user can interact with the user devices 110(1)-110(N) via an external device (not shown; e.g., a laptop, a smartphone, etc.). For example, the user may configure settings the user devices 110(1)-110(N) through the external device (e.g., turn one or more of the user devices 110(1)-110(N) on/off, change a sampling rate, etc.). In some cases, a user may further be able to provide feedback relating to one or more predictions generated using the computer system 130 or manually report health information to the computer system 130 via the user devices 110(1)-110(N). For example, in some cases, the user may, e.g., through one or more of the user devices 110(1)-110(N), report to the computer system 130 that they have the flu or an influenza-like illness, which may be used by the computer system 130 in training models to recognize features of received physical statistical data indicative of certain health conditions.

Each user of the user devices 110(1)-110(N) may be a member of a population monitored by the computer system 130. In some cases, each user is associated with one or more of the user devices 110(1)-110(N) measuring physical statistics of that user. For example, the user devices 110(1)-110(N) can measure a user's resting heart rate (RHR) over time, a daily number of steps (or other measure of activity level such as distance walked), and sleep statistics (such as duration of sleep, number of times sleep was interrupted, sleep start and end times, etc.) for the user. Recorded physical statistics from the user devices 110(1)-110(N) may be stored as physical statistic data and sent by the user devices 110(1)-110(N) to the computer system 130 for analysis. In some cases, some or all physical statistic data is collected as time series data, or periodically recorded measurements of physical statistics of the user over time. The frequency of measurements included in the physical statistics data sent to the computer system 130 can depend on the user devices 110(1)-110(N), user preference selections, or the type of physical statistic data being collected. For example, the user devices 110(1)-110(N) may send time series data for average RHR multiple times per day, but send hours slept data once per day. In some cases, the user devices 110(1)-110(N) may send physical statistic data to the computer system 130 frequently, for example, hourly or in real time.

The user devices 110(1)-110(N) (and, in some cases, users of the user devices 110(1)-110(N)) may communicate with the computer system 130 over the network 120. The network 120 may be a network or system of networks connecting the computer system 130 to the user devices 110(1)-110(N). The network 120 may comprise any combination of local area or wide area networks, using wired or wireless communication systems. In some cases, the network 120 uses standard communications technologies or protocols. For example, the network 120 can include communication links using technologies such as Ethernet, 3G, 4G, CDMA, WIFI, and Bluetooth. Data or information exchanged over the network 120 may be represented using any suitable format, such as hypertext markup language (HTML) or extensible markup language (XML). In some cases, all or some of the communication links of the network 120 may be encrypted using any suitable technique or techniques. In some implementations, the network 120 also facilitates communication between the computer system 130, the user devices 110(1)-110(N), and other entities of the environment 100 such as the modeling unit 135, users (not shown) or other external devices (not shown).

The computer system 130 may comprise computer devices such as a server, server cluster, distributed server, or cloud-based server capable of predicting health conditions, statistics, risks, etc. For example, the computer system 130, using the modeling unit 135, may predict chronic health conditions (CHC) symptoms, acute illness symptoms, or any other health issue for a user within a population based on physical statistics received from that user. In some cases, the computer system 130 gathers physical statistics about a set of users within a population (e.g., through data from one or more of the user devices 110(1)-110(N)). Physical statistics may be measurements characterizing a user's activity level or current health state (such as from the user devices 110(1)-110(N), or other sources). For example, physical statistics can include measurements of the user's vital signs such as body temperature, resting heart rate (RHR), blood pressure, current heart rate (for example, presented as a time series), heart rate variability, respiration rate, or galvanic skin response, measurements of user activity such as daily number of steps, distance walked, time active, or exercise amount, sleep statistics such as time slept, number of times sleep was interrupted, or sleep start and end times, or other similar metrics.

The computer system 130 can analyze received physical statistic data to extract learned features or generate a learned representation of the physical statistic data using the modeling unit 135. In some cases, the learned representation generated by the modeling unit 135 may store a transformed, modified, or compressed version of raw physical statistic data. This version of the raw physical statistic data (or wearable device data) may preserve richness of information and useful features that may be used to identify trends or outliers among data gathered across a large population, predict health conditions, segment, cluster, or categorize data from different users, or the like.

In some cases, outputs (e.g., predictions of risk of an acute illness to a user or a population) from the computer system 130 may be shared. For example, the outputs from the computer system 130 may be shared with the corresponding user. In another example, the outputs from the computer system 130 may be shared with researchers or a laboratory (e.g., for studying infectious diseases), with consent from the corresponding user. In another example, the outputs from the computer system 130 may be shared with healthcare providers (e.g., the corresponding user's primary care physician), with consent from the corresponding user. In cases where the outputs from the computer system 130 are shared with the healthcare providers, the healthcare providers may maintain a computing system such as a server, set of servers, server cluster, etc. which can create or modify an individual treatment plan or perform interventions based on predicted health conditions generated using the computer system 130. For example, the computing system of the healthcare providers can be managed by a medical provider, doctor, or other entity providing medical care to a user for a health condition.

### Example Methods

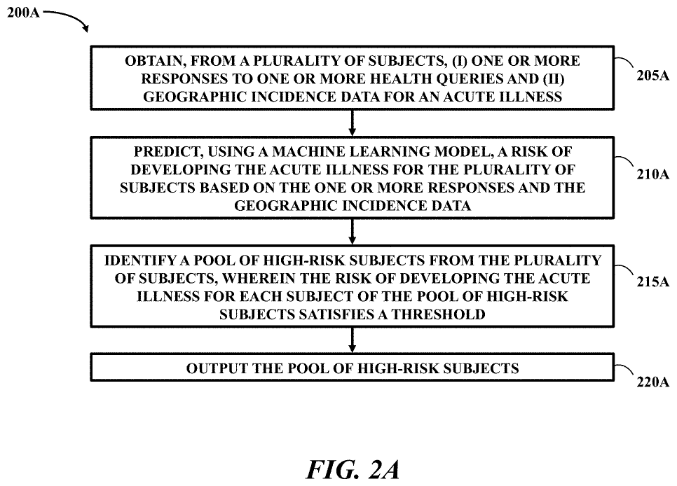

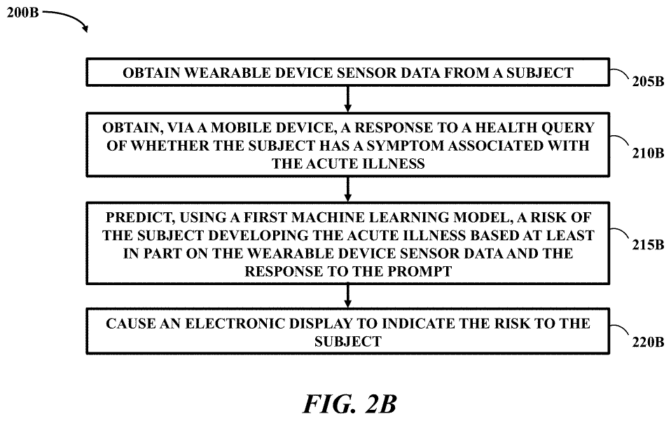

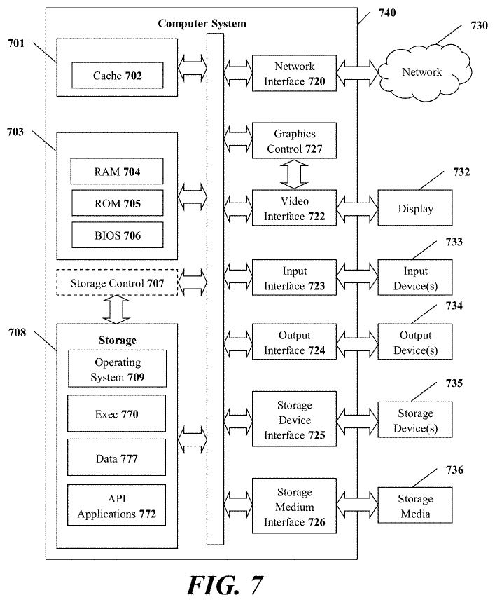

FIG. 2A is a flowchart illustrating an example method 200A for assembling a pool of high-risk subjects for developing an acute illness, in accordance with some embodiments. The method 200A may be implemented using one or more systems (e.g., hardware or software) described herein (e.g., the environment 100). The method 200A may implement one or more techniques described herein (e.g., the workflow 400). At a high level, the method 200A may include obtaining, from a plurality of subjects, (i) one or more responses to one or more health queries, and (ii) geographic incidence data for an acute illness (block 205A); predicting, using a machine learning model, a risk of developing the acute illness for the plurality of subjects based on the one or more responses and the geographic incidence data (block 210A); identifying a pool of high-risk subjects from the plurality of subjects, wherein the risk of developing the acute illness for each subject of the pool of high-risk subjects satisfies a threshold (block 215A); and outputting the pool of high-risk subjects (block 220A). The method 200A may be implemented using systems that may be the same as or similar to the systems of the environment 100 of FIG. 1, or the computer system 700 of FIG. 7. One or more operations in the method 200B may be performed on a repeating or iterative basis, for example, at some time interval or frequency.

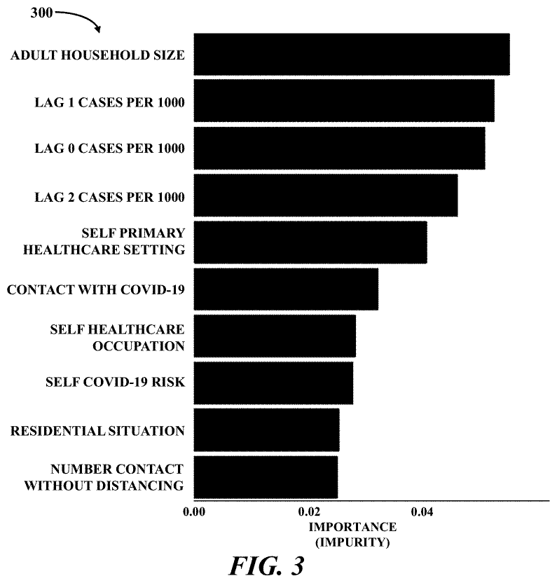

In some cases, the method 200A may begin with obtaining, from the plurality of subjects (i) the one or more responses to the health queries, and (ii) the geographic incidence data for the acute illness at the block 205A. The health queries may include one or more of the health queries described with respect to FIG. 3, or queries of a similar nature. In general, the health queries may be used to assess a subject's, zone's, population's, etc. risk of an acute illness. For example, the health queries may comprise one or more of a household composition query, an occupation query, a residence query, or an infected contact query. The responses to the one or more health queries may be obtained at a computer system. For example, the responses may be obtained at a mobile device, such as a smartphone or a wearable device. The responses may include text data, video data, audio data, etc. The responses may include physiological data that includes one or more of resting heart rate data, sleep data, step count data, blood pressure data, caloric data, nutrition data, or body temperature data. The geographic incidence data may generally be data about the spread of an acute illness over a geographic area. For example, the geographic incidence data may be over a continent, a country, a region, a state, a province, a county, a jurisdiction, a zip code, a city, a township, a village, a neighborhood, a street, a block, a property, etc. In some cases, the geographic incidence data is associated with a plurality of zip code tabulation areas (ZCTAs).

In some cases, the method 200A may include predicting, using the machine learning model, the risk of developing the acute illness for the plurality of subjects based on the one or more responses and the geographic incidence data at the block 210A. In general, the machine learning model may be built to predict risk for the plurality of subjects developing the acute illness. In some cases, the machine learning model may be a decision tree algorithm. In some cases, the machine learning model may be a random forest model. In some cases, the machine learning model may be a generative additive model (GAM). In some cases, the machine learning model may be built using county-level incidence rates as the geographic incidence data. In some cases, the county-level incidence rates may be reported by a news organization or government entity. Over a period of time, such as a number of weeks, a three-dimensional GAM may be fit to the geographic incidence data, with the geographic positioning (e.g., latitude and longitude of the county centroid, borders of the county, etc.) and week number as predictors. The mean squared error of the machine learning model's predictions for weeks following the training period may be calculated across the following hyperparameters: smoother for the longitude and latitude (tensor product or cubic regression with shrinkage) and the rank of the smooths (4, 6, or 8). In some cases, the best-performing machine learning model uses a rank-6 temporal product smoother for the spatial components, and a rank-4 cubic regression spline with shrinkage for week number. These hyper-parameters may then be used to build a series of machine learning models that predict the incidence rate for each ZCTA and week number of the response period using county-level incidence rate for the preceding 10 weeks. When applied to a COVID-19 data set, for each observation, three predictors were added using these models: the predicted COVID-19 incidence rate for the respondents' home ZCTA at the week of diagnosis, and the predictions for the preceding two weeks. In some cases, respondents who did not report a COVID-19 diagnosis may be assigned a random “week of diagnosis” for this purpose. The machine learning model described herein can be used to identify high risk individuals, to identify high risk zones, or a combination of the two. These high-risk individuals and high-risk zones can be prioritized for vaccine distribution, can be flagged for preventative mandates (e.g., masks, school/business closures, etc.), or can be notified in advance of potential outbreaks (e.g., via notification transmission via SMS or emergency communication channel). The machine learning model described herein can also be used to recruit individuals for participation in studies or trials, as described below.

In some cases, the method 200A may include identifying the pool of high-risk subjects from the plurality of subjects, wherein the risk of developing the acute illness for each subject of the pool of high-risk subjects satisfies the threshold at the block 215A. In some cases, the threshold for each subject of the pool of high-risk subjects may be a likelihood (e.g., percentage) that the subject will contract the acute illness. For example, the pool of high-risk subjects may include any number of subjects, provided each of the subjects has a certain likelihood (e.g., 70% likelihood over the next month) of contracting the acute illness. In other cases, the pool of high-risk subjects may have a specified number (e.g., a maximum, minimum, a target number, etc.) of subjects to be included in the pool of high-risk subjects. For example, the pool of high-risk subjects may have a threshold such that the 100 subjects with the highest risk are identified to be in the pool of high-risk subjects.

In some cases, the method 200A may include outputting the pool of high-risk subjects at the block 220A. The output may include identifiers (e.g., name, contact information, identification number, etc.) of the subjects identified in the pool of high-risk subjects. Outputting the pool of high-risk subjects may include providing the output (e.g., the identifiers) as text, audio, video, image, or the like. For example, the output may be displayed on a graphical user interface. In some cases, the output may be stored (e.g., in a memory or database) for later use.

In some cases, after identifying the pool of high-risk subjects (or “high-risk pool”), the method 200A may include predicting an incidence of the acute illness for a population (e.g., for a population comprising the pool of high-risk subjects or for a population similar in at least one characteristic common to the pool of high-risk subjects). Predicting the incidence rate for the acute illness for the population may comprise processing wearable device sensor data, demographic data, and/or geographic data with a second machine learning model that may be different from the machine learning model used for generating the high-risk pool. Alternatively, predicting the incidence rate for the acute illness for the population may comprise processing data with the same machine learning model (e.g., applied in a different manner) as the machine learning model used generating the high-risk pool. The incidence rate over the population may be predicted for a past, present, or future time period.

In some cases, after identifying the pool of high-risk subjects, the method 200A may include conducting a study for a vaccine for the acute illness by a population (e.g., a population comprising the pool of high-risk subjects). As described in the Summary section, when conducting a vaccine trial (e.g., a phase II or phase III trial), vaccine efficacy may be determined based on comparing infection rates between subjects with the placebo and subjects with the actual vaccine. An effective vaccine may be a vaccine for which the subjects with the placebo are infected at a higher rate than the subjects with the vaccine, where the higher rate is statistically significant. By enriching the vaccine trial with more subjects who are likely to become infected, the overall pool of subjects recruited for the vaccine trial may be smaller. For example, if a study that may have otherwise had 10,000 subjects (e.g., to achieve statistically significant results) could be enriched by a factor of 2 (as in, the pool of subjects are twice as likely as the control pool to become infected), then the study may only need to recruit 5,000 subjects. Conducting the vaccine study may comprise processing wearable device sensor data, demographic data, and/or geographic data with a second machine learning model that may be different from the machine learning model used for generating the pool of high-risk subjects. Alternatively, conducting the vaccine study may comprise processing data with the same machine learning model (e.g., applied in a different manner) as the machine learning model used for generating the high-risk pool. The vaccine study may be conducted for a past, present or future time period.

FIG. 2B is a flowchart illustrating an example method 200B for presenting information about an acute illness to a subject, in accordance with some cases. The method 200B may be implemented using one or more systems (e.g., hardware or software) described herein (e.g., the environment 100). The method 200B may implement one or more techniques described herein (e.g., the workflow 400). At a high level, the method 200B may include obtaining wearable device sensor data from the subject (block 205B); obtaining, via a mobile device, a response to a health query of whether the subject has a symptom associated with the acute illness (block 210B); predicting, using a machine learning model, a risk of the subject developing the acute illness based at least in part on the wearable device sensor data and the response to the health query (block 215B); and causing an electronic display to indicate the risk to the subject (block 220B). The method 200B may be implemented using systems that may be the same as or similar to the systems of the environment 100 of FIG. 1, or the computer system 700 of FIG. 7. One or more operations in the method 200B may be performed on a repeating or iterative basis, for example, at some time interval or frequency.

In some cases, the method 200B may begin with obtaining wearable device sensor data from the subject at the block 205B. The wearable device sensor data may be collected via one or more wearable devices worn by the subject. For example, the wearable devices may be smartwatches (e.g., the user device 110(1) of FIG. 1), smartbands (e.g., a Fitbit® device), smartclothing, smartshoes, or the like. The wearable device sensor data may include physiological data that includes one or more of resting heart rate data, sleep data, step count data, blood pressure data, caloric data, nutrition data, or body temperature data. The wearable device sensor data may include physical statistics of the subject. In some cases, the physical statistics can include measurements of the subject's vital signs such as body temperature, resting heart rate (RHR), blood pressure, current heart rate (for example, presented as a time series), heart rate variability, respiration rate, or galvanic skin response, measurements of user activity such as daily number of steps, distance walked, time active, or exercise amount, sleep statistics such as time slept, number of times sleep was interrupted, or sleep start and end times, or other similar metrics. The wearable device may present the wearable device data to the subject using, for example, a graphical user interface or speakers. The wearable device may collect (and, in some cases, provide to a machine learning model) the wearable device sensor data on a repeating basis (e.g., at some time interval or frequency).

In some cases, the method 200B may include obtaining, via the mobile device, the response to the health query of whether the subject has the symptom associated with the acute illness at the block 210B. Similar to the health queries described with respect to the block 205A, the health query may ask a subject directly (e.g., “Do you have symptoms of COVID-19?”, etc.) or indirectly (e.g., “Do you have a fever?”, “Do you feel tired?”, etc.) if they have symptoms associated with the acute illness. In some cases, the response from the subject may be a string. The string may be interpreted (e.g., categorized) by algorithms. The response may be selected (e.g., by a user) from a series of options (e.g., multiple choice options for different careers). The response may be positive or negative (e.g., yes or no). The mobile device may be, in some cases, a smartphone or a tablet. The mobile device may be, in some cases, a wearable device. In some cases, the wearable device may be the same wearable device as the wearable device described with respect to the block 205B that collects the wearable device sensor data. The health queries may be presented (and the responses may be provided to a machine learning model) on a repeating basis (e.g., at some time interval or frequency).

In some cases, the method 200B may include predicting, using the machine learning model, the risk of the subject developing the acute illness based at least in part on the wearable device sensor data and the response to the health query at the block 215B. In general, the machine learning model may leverage the wearable device sensor data to identify the high-risk subjects at the right time, and possibly match them with an according action. In some cases, the machine learning model may be a decision tree algorithm. In some cases, the machine learning model may be a random forest model. In some cases, the machine learning model may be a GAM. In some cases, the machine learning model may be the same as or similar to, in at least some aspects, the machine learning model described with respect to the block 210A.

In some cases, the method 200A may include causing the electronic display to indicate the risk to the subject at the block 220B. The indication may include a likelihood or timeframe that the subject will contract the acute illness. For example, the electronic display (e.g., of the mobile device, of the wearable device, etc.) may indicate that the subject has a 50% chance of testing positive for the flu within the next 72 hours. In some cases, the indication may be presented via audio, video, haptic, or other suitable techniques to the subject. In some cases, the indication may be stored (e.g., in a memory or database) for later use.

### Example Feature Importance

FIG. 3 shows a non-limiting example of data 300 illustrating feature importance of different variables. The variables may be responses to one or more health queries. Specifically, the data 300 illustrates feature importance for predicting an incidence rate of an acute illness in a pool of subjects, where the acute illness is COVID-19 and the prediction is made via a random forest machine learning model. As illustrated, feature importance is measured as the total decrease in node impurities (measured with the Gini index) from splitting on that variable. In descending order of importance, the data 300 includes variables of adult household size, lag 1 cases per 1000, lag 0 cases per 1000, lag 2 cases per 1000, self primary healthcare setting, contact with COVID-19, self healthcare occupation, self COVID-19 risk, residential situation, and number contact without distancing.

The most important variable, as illustrated, is adult household size. The adult household size variable may be an integer that is the response, from each of the subjects in the pool to a question, “How many adults (age 18+) currently live in your household (including yourself)? Include roommates, or if you are living in a group home, the total number of adults you share common living spaces with.”

The variables Lag (0, 1, 2), may correspond to a series of hotspot prediction models built using county-level prevalence data provided by public data sources (e.g., government entity, news outlets, etc.). A three-dimensional generative adversarial model (GAM) may be fit to prevalence values with latitude, longitude, and (week number) as predictors, for weeks 1-N. In some cases, this model may predict for week N+1. For each observation used to train the model, the predicted prevalence rate may be determined for the associated zip code for the week number of the response and the preceding two weeks.

The variable self primary healthcare setting may correspond to a response (e.g., subject response) to, “In which healthcare setting do you primarily work in?” The variable contact with COVID-19 may correspond to a response (e.g., subject response) to, “Have you been in prolonged close contact (e.g., within 6 feet for at least 10 minutes) with someone who was diagnosed with COVID-19?” The variable self healthcare operation may correspond to a response (e.g., subject response) to, “Do you work in one of the following healthcare-related occupations?” The variable self COVID-19 risk may correspond to a response (e.g., subject response) to, “What do you believe is your personal risk of getting COVID-19? Please think about your job, your usual activities, your close contacts etc.” The variable residential situation may correspond to a response (e.g., subject response) to, “What is your current residential situation?” The variable number contacted without distancing may correspond to a response (e.g., subject response) to “On an average workday, how many individuals do you closely interact with that do not follow social distancing recommendations (e.g., have a face-to-face conversation, help with a task and do not stay at least 6 feet apart)?” One or more of the responses to the above questions may be a string. The string may be interpreted (e.g., categorized) by algorithms. One or more of the responses to the above questions may be selected (e.g., by a user) from a series of options (e.g., multiple choice options for different careers). One or more of the responses to the above questions may be positive or negative (e.g., yes or no).

### Example Workflows

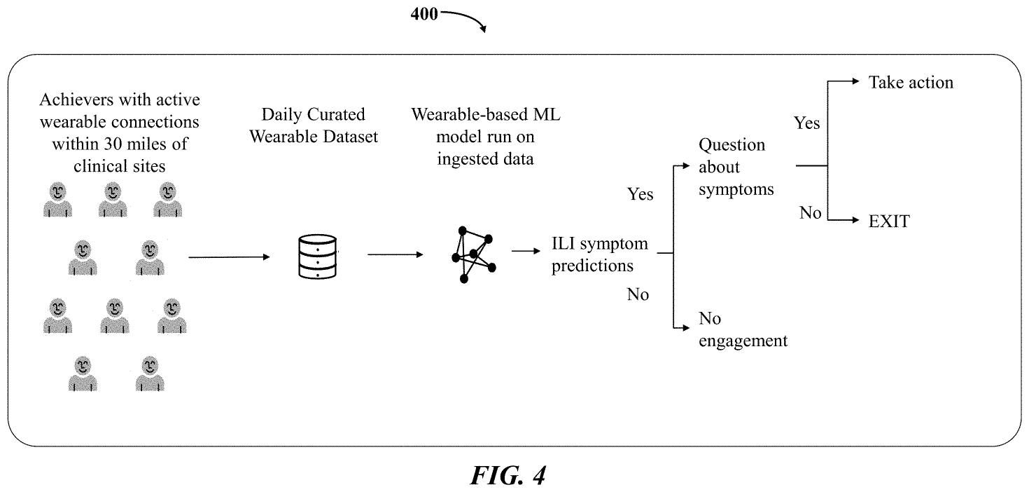

FIG. 4 shows an example of a workflow 400 for collecting data that may be used in conjunction with systems, methods, computer-readable media, or techniques described herein. At a high level, the workflow 400 may be used for assembling a pool of high-risk subjects for developing an acute illness or for presenting information to a subject about an acute illness, such as described with respect to the method 200A of FIG. 2A and method 200B of FIG. 2B, respectively. The workflow 400 may be implemented using systems that may be the same as or similar to the systems of the environment 100 of FIG. 1, or the computer system 700 of FIG. 7.

In some cases, the workflow 400 may begin with ingesting wearable device sensor data from a plurality of users. As illustrated, the users may be nearby a clinical site. For example, the wearable device sensor data may be ingested from users who are within 30 miles of a clinical site, as illustrated. In some cases, the users may be any distance from a clinical site.

The wearable device sensor data may be ingested at some frequency. For example the wearable device sensor data may be ingested hourly, twice a day, daily, weekly, monthly, or some other suitable frequency. As illustrated, the wearable device sensor data may be ingested daily. The wearable device sensor data may be collected via one or more wearable devices worn by a user. For example, the wearable devices may be smartwatches (e.g., the user device 110(1) of FIG. 1), smartbands (e.g., a Fitbit® device), smartclothing, smartshoes, or the like. The wearable device sensor data may include physiological data that includes one or more of resting heart rate data, sleep data, step count data, blood pressure data, caloric data, nutrition data, or body temperature data. The wearable device sensor data may include physical statistics of the user. In some cases, the physical statistics can include measurements of the user's vital signs such as body temperature, resting heart rate (RHR), blood pressure, current heart rate (for example, presented as a time series), heart rate variability, respiration rate, or galvanic skin response, measurements of user activity such as daily number of steps, distance walked, time active, or exercise amount, sleep statistics such as time slept, number of times sleep was interrupted, or sleep start and end times, or other similar metrics. The wearable device may present the wearable device data to the user using, for example, a graphical user interface or speakers.

Once ingested, the wearable device sensor data may be provided to a machine learning model for analysis. The machine learning model may predict the risk of each user developing an acute illness (e.g., influenza-like illness, as illustrated) based at least in part on the wearable device sensor data. In general, the machine learning model may leverage the wearable device sensor data to identify the high-risk subjects at the right time, and possibly match them with an according action (e.g., refer them to a nearby clinical site). In some cases, the machine learning model may be a decision tree algorithm. In some cases, the machine learning model may be a random forest model. In some cases, the machine learning model may be a generative additive model. In some cases, the machine learning model may be the same as or similar to, in at least some aspects, the machine learning model described with respect to the block 210A or the block 210B.

If the machine learning model predicts that a user has symptoms consistent with the acute illness (e.g., influenza-like illness, as illustrated), then the user may be prompted with additional health queries. The additional health queries may be about risk factors (e.g., household size and residential situation, location, occupation, etc.), about physiological data (e.g., resting heart rate data, sleep data, step count data, blood pressure data, caloric data, nutrition data, or body temperature data), or about symptoms (e.g., fever, cough, congestion, aches, nausea, etc.).

In some cases, if a user does not answer the additional health queries, the workflow 400 may exit. In some cases, if a user does provide answers to the additional health queries, but the answers suggest that the user does not have, or is not at a high risk of having, the acute illness, the workflow 400 may exit. Similarly, in some cases, if the machine learning model predicts that a user does not have symptoms consistent with the acute illness, the workflow 400 may exit rather than prompt the user with the additional health queries.

In cases where the workflow 400 continues (does not exit), the workflow 400 may take action. The action may include enabling the user to enroll in a study or trial (e.g., at a nearby clinical site). The action may include alerting the user that they have a high risk of having the acute illness (e.g., via a graphical user interface). The action may include suggesting medication (e.g., over the counter, behind-the-counter, prescription, non-traditional, etc.) or behavior (e.g., resting, eating fruit, apply cold or hot compresses, etc.) to help reduce the effects of, or prevent, the acute illness. The action may include contacting a medical provider on behalf of the user. The action may include sharing (with user consent) the wearable device sensor data with the study or trial or with the medical provider. The action may include sharing (with user consent) the acute illness risk prediction with the study or trial or with the medical provider.

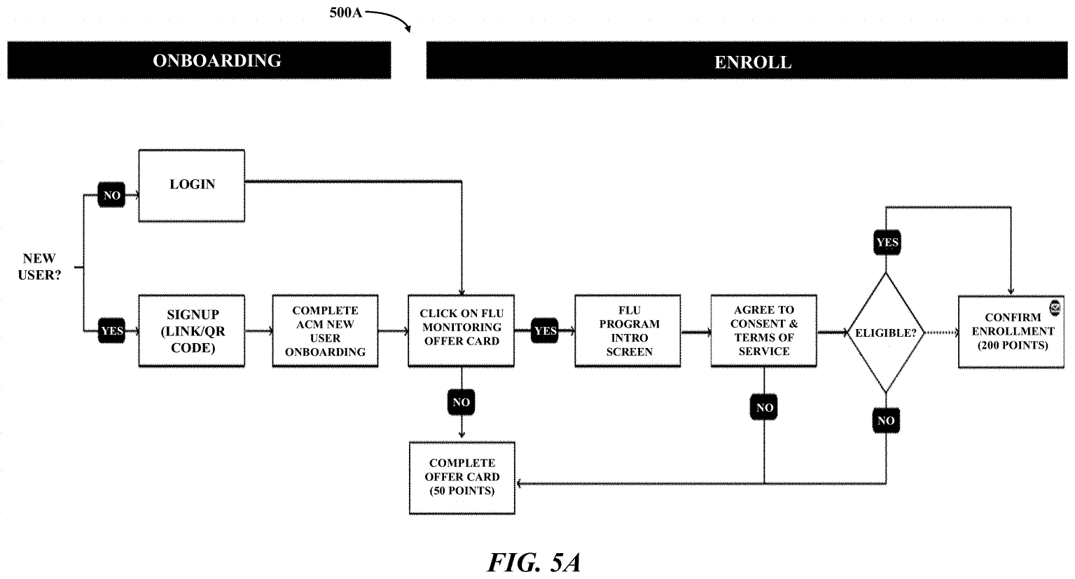

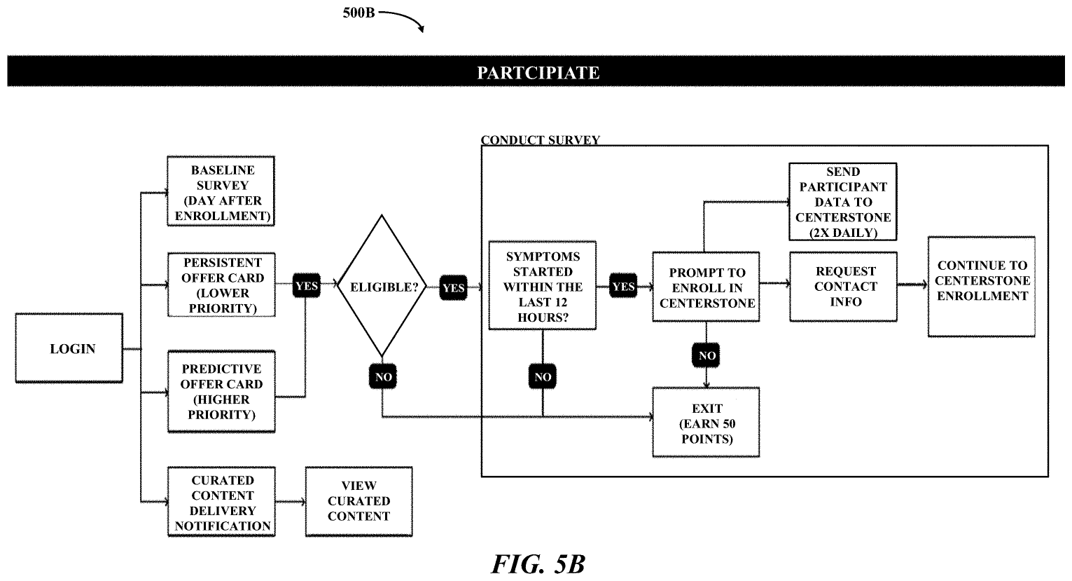

FIG. 5A shows an example of a workflow 500A for onboarding and enrolling users that may be used in conjunction with systems, methods, computer-readable media, or techniques described herein. At a high level, the workflow 500A may be used for assembling a pool of high-risk subjects for developing an acute illness or for presenting information to a subject about an acute illness, such as described with respect to the method 200A of FIG. 2A and method 200B of FIG. 2B, respectively. The workflow 500A may be implemented using systems that may be the same as or similar to the systems of the environment 100 of FIG. 1, or the computer system 700 of FIG. 7.

In some cases, the workflow 500A may begin with onboarding a new user or enabling a returning user to login to their account. Onboarding a new user may include obtaining information (e.g., health history, demographics, contact information, biographical information, occupation, residential information, etc.) of the user. Once a user is onboarded and logged in, the user may be able to enroll in a study or trial. The study or trial may be specific to an acute illness, such as influenza-like symptoms, as illustrated.

To enroll the user, in some cases, the workflow 500A may include providing information about the acute illness or the process predicting risk of the acute illness. The user may agree to consent and terms of service in the workflow 500A. For example, the user may agree to collection of certain types of data (e.g., wearable device sensor data), sharing certain types of data (e.g., with the trial or study, with healthcare providers, etc.), etc. The workflow 500A may further include determining if the user is eligible to participate in the study or trial. Eligibility may be based on demographics, symptoms, size of the study or trial, health history, or other suitable factors. If the user is eligible, the user may confirm enrollment in the study or trial in the workflow 500A.

FIG. 5B shows an example of a workflow 500B for user participation that may be used in conjunction with systems, methods, computer-readable media, or techniques described herein. At a high level, the workflow 500B may be used for assembling a pool of high-risk subjects for developing an acute illness or for presenting information to a subject about an acute illness, such as described with respect to the method 200A of FIG. 2A and method 200B of FIG. 2B, respectively. The workflow 500B may be implemented using systems that may be the same as or similar to the systems of the environment 100 of FIG. 1, or the computer system 700 of FIG. 7. The workflow 500B may be executed in response to a user being enrolled in a trial or study, such as described with respect to the workflow 500A. One or more aspects of the workflow 500B may be the same as or similar to the workflow 500A.

In some cases, the workflow 500B may begin with a user logging in to an account. The user may be able to complete a baseline survey after logging in. The baseline survey may obtain information (e.g., health history, demographics, contact information, biographical information, occupation, residential information, etc.) of the user while the user is not experiencing one or more acute illnesses. The user may be able to view curated content (e.g., the same as or similar to the user interfaces 600A-600F) based on information about the user. One or more offer cards (e.g., the same as or similar to the user interfaces 600A-600F) may be presented to the user at the workflow 500B. The offer cards may prompt the user to participate in a study or trial.

In some cases, if the user is eligible (e.g., based on demographics, symptoms, size of the study or trial, health history, or other suitable factors) to participate in the study or trial, the workflow 500B may conduct a survey. The user may be prompted about how long ago their symptoms started. Also or alternatively, wearable device sensor data may be analyzed to determine how long ago symptoms started. In any case, if the symptoms started longer than a threshold amount of time ago, the workflow 500B may exit. For example, with faster developing illnesses the threshold amount of time may be shorter than for slower developing illnesses. In some examples, the threshold amount of time may be 1 hour, 4 hours, 6 hours, 12 hours, 18 hours, 24 hours, 36 hours, 2 days, 4 days, one week, two weeks, one month, 3 months, 6 months, one year, etc.

Provided the user's symptoms have started in an amount of time that satisfies the threshold, the user may be prompted to enroll in the study or trial. In the study or trial, the workflow 500B may include sending (with user consent) data (e.g., wearable device sensor data, responses to health queries, demographic information, health history information, symptom data, etc.) to the study or trial. Further, the workflow 500B may include participating in the study or trial that may implement the systems, the methods, the computer-readable media, or the techniques described herein. For example, the study or trial may implement methods that are the same as or similar to the method 200A or the method 200B.

### Example User Interfaces

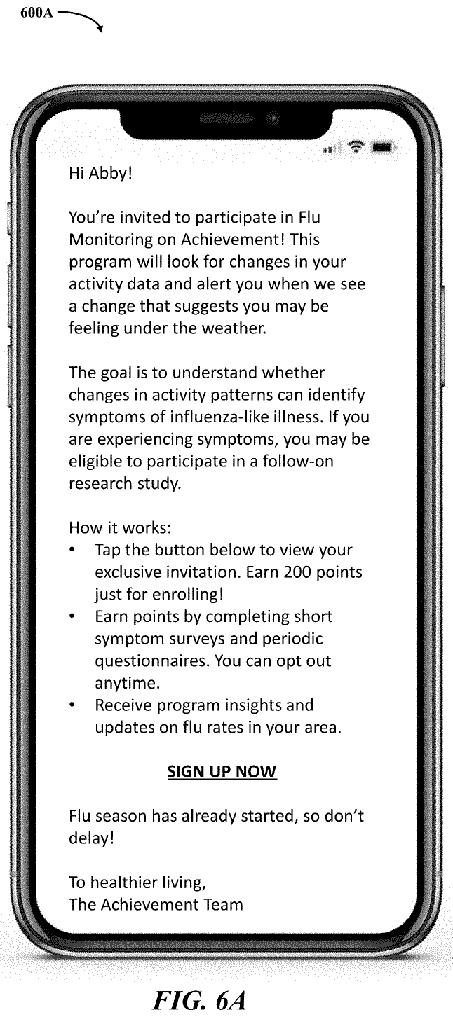

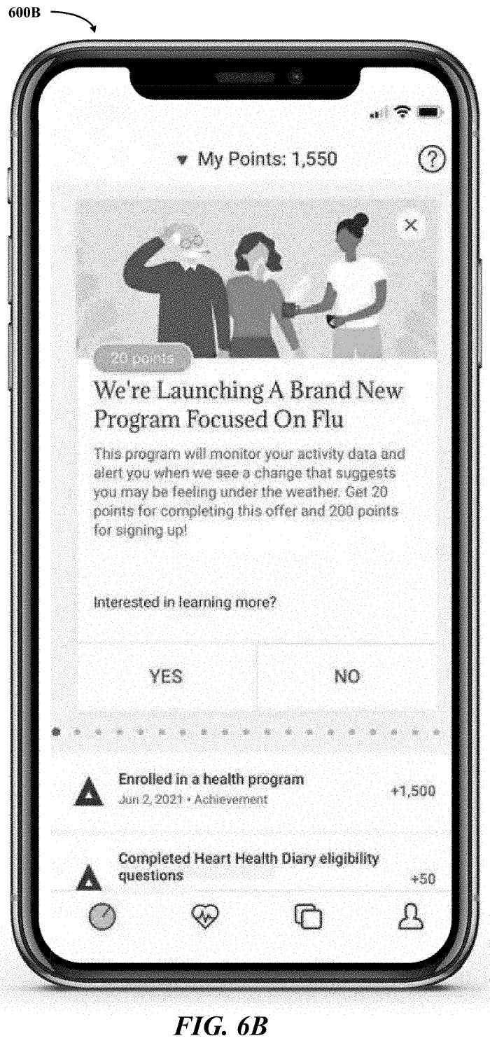

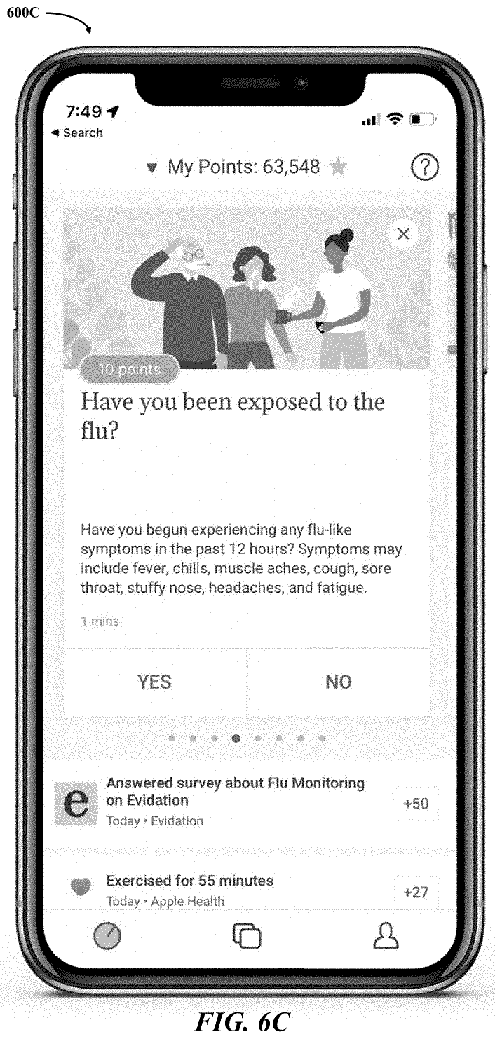

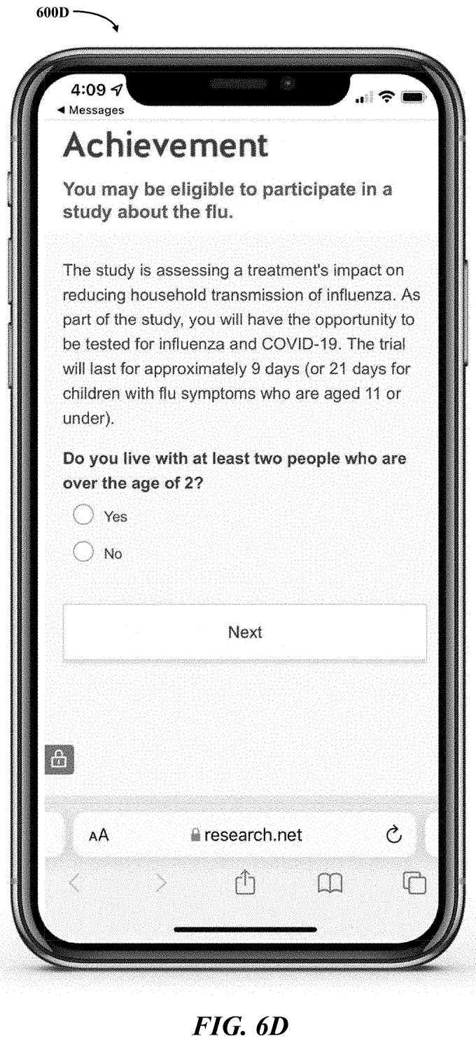

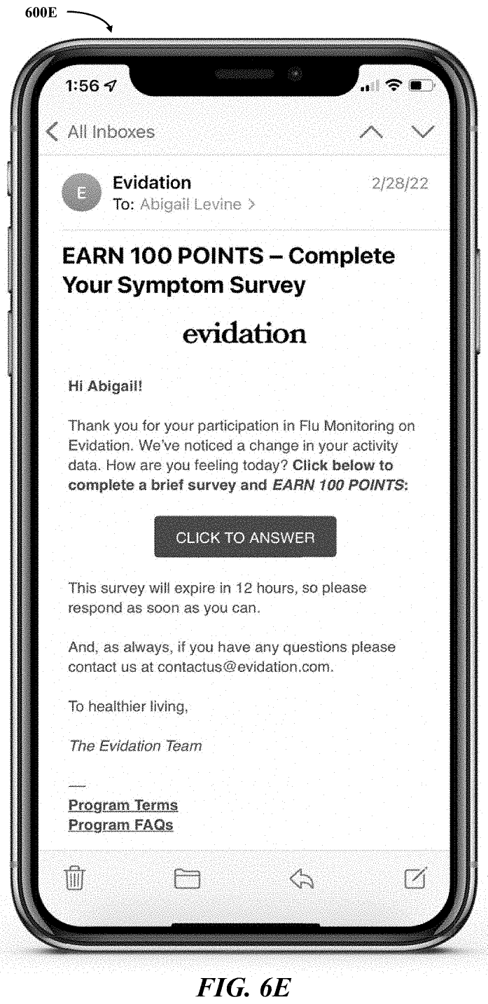

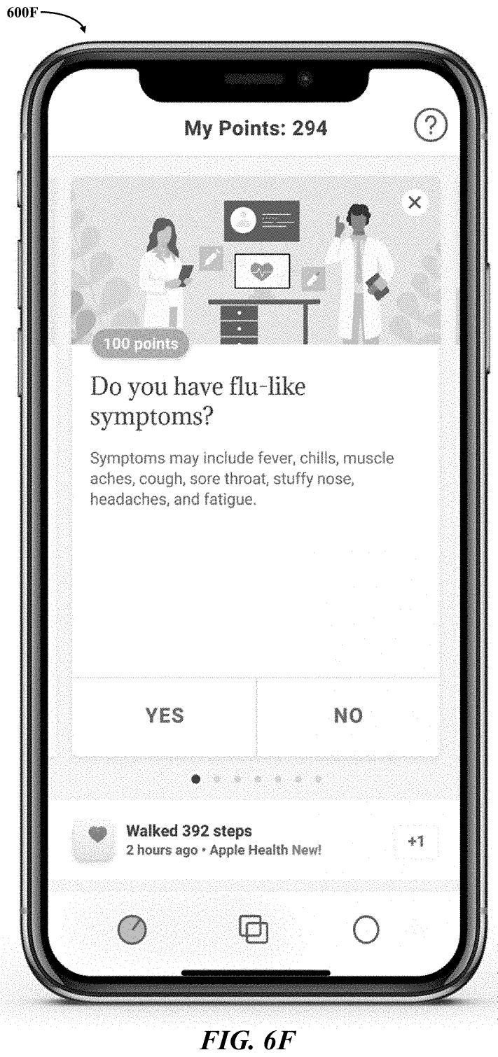

The systems, the methods, the computer-readable media, and the techniques described herein may be implemented at least in part with the use of a user interface that may be presented on a graphical user interface (e.g., the display 732 of FIG. 7). FIGS. 6A-6F illustrate example user interfaces 600A-600F. At a high level, the user interfaces 600A-600F may be used for assembling a pool of high-risk subjects for developing an acute illness or for presenting information to a subject about an acute illness.

FIG. 6A shows the example user interface 600A of an enrollment interface. As illustrated, the user interface 600A includes text explaining example techniques for identifying acute illnesses (e.g., influenza-like illnesses, as illustrated). The user interface 600A may be accessible from a mobile computing device (e.g., a smartphone, a tablet, a wearable device, etc.). The user interface 600A may be displayed in response to opening an email, a text, an application, etc. The user interface 600A may be accessible from a non-mobile computing device (e.g., a desktop computer). The user interface 600A, includes an enrollment function (e.g., the “SIGN UP NOW” button, as illustrated). In selecting the enrollment function, a user may initiate enrollment in one or more of the systems, the methods, the computer-readable media, or the techniques described herein. For example, in selecting the enrollment function, the user may initiate the workflow 500A for onboarding and enrolling users.

FIG. 6B shows the example user interface 600B of an enrollment interface. As illustrated, the user interface 600B includes text explaining example techniques for identifying acute illnesses (e.g., influenza-like illnesses, as illustrated). The user interface 600B may be accessible from a mobile computing device (e.g., a smartphone, a tablet, a wearable device, etc.). The user interface 600B may be displayed in response to opening an email, a text, an application, etc. The user interface 600B may be accessible from a non-mobile computing device (e.g., a desktop computer). The user interface 600B, includes an enrollment function (e.g., the “YES” button, as illustrated). In selecting the enrollment function, a user may initiate enrollment in one or more of the systems, the methods, the computer-readable media, or the techniques described herein. For example, in selecting the enrollment function, the user may initiate the workflow 500A for onboarding and enrolling users.

FIG. 6C shows the example user interface 600C of a participation interface. As illustrated, the user interface 600C includes text displaying a health query of whether a subject has a symptom associated with an acute illness (e.g., flu-like symptoms, as illustrated). The user interface 600C may be accessible from a mobile computing device (e.g., a smartphone, a tablet, a wearable device, etc.). The user interface 600C may be displayed in response to opening an email, a text, an application, etc. The user interface 600C may be accessible from a non-mobile computing device (e.g., a desktop computer). The user interface 600C, includes a multiple choice response function (e.g., the “YES” or “NO” button, as illustrated). In selecting one of the multiple choice response functions, a user may participate in one or more of the systems, the methods, the computer-readable media, or the techniques described herein. For example, selecting one of the options in the multiple choice response function may be included in the workflow 500B for user participation.

FIG. 6D shows the example user interface 600D of a participation interface. As illustrated, the user interface 600D includes text displaying a health query of whether a subject lives with at least two people who are over the age of two. The user interface 600D may be accessible from a mobile computing device (e.g., a smartphone, a tablet, a wearable device, etc.). The user interface 600D may be displayed in response to opening an email, a text, an application, etc. The user interface 600D may be accessible from a non-mobile computing device (e.g., a desktop computer). The user interface 600D, includes a multiple choice response function (e.g., the “YES” or “NO” button, as illustrated). In selecting one of the multiple choice response functions, a user may participate in one or more of the systems, the methods, the computer-readable media, or the techniques described herein. For example, selecting one of the options in the multiple choice response function may be included in the workflow 500B for user participation and may initiate prompting of additional health queries.

FIG. 6E shows the example user interface 600E of a participation interface. As illustrated, the user interface 600E includes text indicating a change in a user's behavior (e.g., based on wearable device sensor data). Further, the user interface 600E includes a view health query function enabling the user to access one or more health queries (e.g., regarding symptoms). The user interface 600E may be accessible from a mobile computing device (e.g., a smartphone, a tablet, a wearable device, etc.). The user interface 600E may be displayed in response to opening an email, a text, an application, etc. The user interface 600E may be accessible from a non-mobile computing device (e.g., a desktop computer). The user interface 600E, includes a multiple choice response function (e.g., the “YES” or “NO” button, as illustrated). In selecting the view health query function (e.g., the “CLICK TO ANSWER” button, as illustrated), the user may initiate presentation of one or more health queries in accordance with in one or more of the systems, the methods, the computer-readable media, or the techniques described herein. For example, in selecting the view health query function, the user may initiate the workflow 500B for user participation.

FIG. 6F shows the example user interface 600F of a participation interface. As illustrated, the user interface 600F includes text displaying a health query of whether a subject has a symptom associated with an acute illness (e.g., flu-like symptoms, as illustrated). The user interface 600F may be accessible from a mobile computing device (e.g., a smartphone, a tablet, a wearable device, etc.). The user interface 600F may be displayed in response to opening an email, a text, an application, etc. The user interface 600F may be accessible from a non-mobile computing device (e.g., a desktop computer). The user interface 600F, includes a multiple choice response function (e.g., the “YES” or “NO” button, as illustrated). In selecting one of the multiple choice response functions, a user may participate in one or more of the systems, the methods, the computer-readable media, or the techniques described herein. For example, selecting one of the options in the multiple choice response function may be included in the workflow 500B for user participation.

### Example Computing System

Referring to FIG. 7, a block diagram is shown depicting an example machine that includes a computer system 700 (e.g., a processing or computing system) within which a set of instructions can execute for causing a device to perform or execute any one or more of the aspects or methodologies for static code scheduling of the present disclosure. The components in FIG. 7 are examples and do not limit the scope of use or functionality of any hardware, software, embedded logic component, or a combination of two or more such components with particular implementations.

Computer system 700 may include one or more processors 701, a memory 703, and a storage 708 that communicate with each other, and with other components, via a bus 740. The bus 740 may also link a display 732, one or more input devices 733 (which may, for example, include a keypad, a keyboard, a mouse, a stylus, etc.), one or more output devices 734, one or more storage devices 735, and various tangible storage media 736. All of these elements may interface directly or via one or more interfaces or adaptors to the bus 740. For instance, the various tangible storage media 736 can interface with the bus 740 via storage medium interface 726. Computer system 700 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 700 includes one or more processor(s) 707 (e.g., central processing units (CPUs), general purpose graphics processing units (GPGPUs), or quantum processing units (QPUs)) that carry out functions. Processor(s) 701 optionally contains a cache memory unit 702 for temporary local storage of instructions, data, or computer addresses. Processor(s) 701 are configured to assist in execution of computer readable instructions. Computer system 700 may provide functionality for the components depicted in FIG. 7 as a result of the processor(s) 701 executing non-transitory, processor-executable instructions embodied in one or more tangible computer-readable storage media, such as memory 703, storage 708, storage devices 735, or storage medium 736. The computer-readable media may store software that implements particular operations, and processor(s) 701 may execute the software. Memory 703 may read the software from one or more other computer-readable media (such as mass storage device(s) 735, 736) or from one or more other sources through a suitable interface, such as network interface 720. The software may cause processor(s) 701 to carry out one or more processes or one or more operations of one or more processes described or illustrated herein. Carrying out such processes or operations may include defining data structures stored in memory 703 and modifying the data structures as directed by the software.

The memory 703 may include various components (e.g., machine readable media) including, but not limited to, a random access memory component (e.g., RAM 704) (e.g., static RAM (SRAM), dynamic RAM (DRAM), ferroelectric random access memory (FRAM), phase-change random access memory (PRAM), etc.), a read-only memory component (e.g., ROM 705), and any combinations thereof. ROM 705 may act to communicate data and instructions unidirectionally to processor(s) 701, and RAM 704 may act to communicate data and instructions bidirectionally with processor(s) 701. ROM 705 and RAM 704 may include any suitable tangible computer-readable media described below. In one example, a basic input/output system 706 (BIOS), including basic routines that help to transfer information between elements within computer system 700, such as during start-up, may be stored in the memory 703.

Fixed storage 708 is connected bidirectionally to processor(s) 701, optionally through storage control unit 707. Fixed storage 708 provides additional data storage capacity and may also include any suitable tangible computer-readable media described herein. Storage 708 may be used to store operating system 709, executable(s) 710, data 711, applications 712 (application programs), and the like. Storage 708 can also include an optical disk drive, a solid-state memory device (e.g., flash-based systems), or a combination of any of the above. Information in storage 708 may, in appropriate cases, be incorporated as virtual memory in memory 703.

In one example, storage device(s) 735 may be removably interfaced with computer system 700 (e.g., via an external port connector (not shown)) via a storage device interface 725. Particularly, storage device(s) 735 and an associated machine-readable medium may provide non-volatile or volatile storage of machine-readable instructions, data structures, program modules, or other data for the computer system 700. In one example, software may reside, completely or partially, within a machine-readable medium on storage device(s) 735. In another example, software may reside, completely or partially, within processor(s) 701.

Bus 740 connects a wide variety of subsystems. Herein, reference to a bus may encompass one or more digital signal lines serving a common function, where appropriate. Bus 740 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 700 may also include an input device 733. In one example, a user of computer system 700 may enter commands or other information into computer system 700 via input device(s) 733. Examples of an input device(s) 733 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some cases, the input device is a Kinect, Leap Motion, or the like. Input device(s) 733 may be interfaced to bus 740 via any of a variety of input interfaces 723 (e.g., input interface 723) including, but not limited to, serial, parallel, game port, USB, FIREWIRE, THUNDERBOLT, or any combination of the above.

In some cases, when computer system 700 is connected to network 730, computer system 700 may communicate with other devices, specifically mobile devices and enterprise systems, distributed computing systems, cloud storage systems, cloud computing systems, and the like, connected to network 730. Communications to and from computer system 700 may be sent through network interface 720. For example, network interface 720 may receive incoming communications (such as requests or responses from other devices) in the form of one or more packets (such as Internet Protocol (IP) packets) from network 730, and computer system 700 may store the incoming communications in memory 703 for processing. Computer system 700 may similarly store outgoing communications (such as requests or responses to other devices) in the form of one or more packets in memory 703 and communicated to network 730 from network interface 720. Processor(s) 701 may access these communication packets stored in memory 703 for processing.

Examples of the network interface 720 include, but are not limited to, a network interface card, a modem, and any combination thereof. Examples of a network 730 or network segment 730 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 730, may employ a wired or a wireless mode of communication. In general, any network topology may be used.

Information and data can be displayed through a display 732. Examples of a display 732 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 732 can interface to the processor(s) 701, memory 703, and fixed storage 708, as well as other devices, such as input device(s) 733, via the bus 740. The display 732 is linked to the bus 740 via a video interface 722, and transport of data between the display 732 and the bus 740 can be controlled via the graphics control 721. In some cases, the display is a video projector. In some cases, the display is a head-mounted display (HMD) such as a VR headset. In further cases, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further cases, the display is a combination of devices such as those disclosed herein.

In addition to a display 732, computer system 700 may include one or more other peripheral output devices 734 including, but not limited to, an audio speaker, a printer, a storage device, and any combinations thereof. Such peripheral output devices may be connected to the bus 740 via an output interface 724. Examples of an output interface 724 include, but are not limited to, a serial port, a parallel connection, a USB port, a FIREWIRE port, a THUNDERBOLT port, and any combinations thereof.

In addition or as an alternative, computer system 700 may provide functionality as a result of logic hardwired or otherwise embodied in a circuit, which may operate in place of or together with software to execute one or more processes or one or more operations of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Various illustrative logical blocks, modules, circuits, and algorithm operations described in connection with the examples disclosed herein may be implemented as electronic hardware, computer software, or combinations of both. To clearly illustrate this interchangeability of hardware and software, various illustrative components, blocks, modules, circuits, and operations have been described above generally in terms of their functionality.

The various illustrative logical blocks, modules, and circuits described in connection with the examples disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The operations of a method or algorithm described in connection with the examples disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An example storage medium may be coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Select televisions, video players, and digital music players with optional computer network connectivity may be suitable for use in the system described herein. Suitable tablet computers, in various cases, include those with booklet, slate, and convertible configurations.

In some cases, the computing device includes an operating system configured to perform executable instructions. The operating system is, for example, software, including programs and data, which manages the device's hardware and provides services for execution of applications. Suitable server operating systems may include, by way of non-limiting examples, FreeBSD, OpenBSD, NetBSD®, Linux, Apple® Mac OS X Server®, Oracle® Solaris®, Windows Server®, and Novell® NetWare®. Suitable personal computer operating systems may include, by way of non-limiting examples, Microsoft® Windows®, Apple® Mac OS X®, UNIX®, and UNIX-like operating systems such as GNU/Linux®. In some cases, the operating system is provided by cloud computing. Suitable mobile smartphone operating systems may include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some cases, the platforms, systems, media, and methods disclosed herein include one or more non-transitory computer readable storage media encoded with a program including instructions executable by the operating system of an optionally networked computing device. In further cases, a computer readable storage medium is a tangible component of a computing device. In still further cases, a computer readable storage medium is optionally removable from a computing device. In some cases, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

In some cases, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. A computer program may be written in various versions of various languages.

The functionality of the computer readable instructions may be combined or distributed in various ways across various environments. In some cases, a computer program comprises one sequence of instructions. In some cases, a computer program comprises a plurality of sequences of instructions. In some cases, a computer program is provided from one location. In some cases, a computer program is provided from a plurality of locations. In some cases, a computer program includes one or more software modules. In some cases, a computer program includes, in part or in whole, one or more web applications, one or more mobile applications, one or more standalone applications, one or more web browser plug-ins, extensions, add-ins, or add-ons, or combinations thereof.

In some cases, a computer program includes a web application. A web application, in various cases, may utilize one or more software frameworks and one or more database systems. In some cases, a web application is created upon a software framework such as Microsoft® .NET or Ruby on Rails (RoR). In some cases, a web application utilizes one or more database systems including, by way of non-limiting examples, relational, non-relational, object oriented, associative, XML, and document oriented database systems. In further cases, suitable relational database systems include, by way of non-limiting examples, Microsoft® SQL Server, mySQL™, and Oracle®. A web application, in some cases, may be written in one or more versions of one or more languages. A web application may be written in one or more markup languages, presentation definition languages, client-side scripting languages, server-side coding languages, database query languages, or combinations thereof. In some cases, a web application is written to some extent in a markup language such as Hypertext Markup Language (HTML), Extensible Hypertext Markup Language (XHTML), or eXtensible Markup Language (XML). In some cases, a web application is written to some extent in a presentation definition language such as Cascading Style Sheets (CSS). In some cases, a web application is written to some extent in a client-side scripting language such as Asynchronous JavaScript and XML (AJAX), Flash® ActionScript, JavaScript, or Silverlight®. In some cases, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some cases, a web application is written to some extent in a database query language such as Structured Query Language (SQL). In some cases, a web application integrates enterprise server products such as IBM® Lotus Domino®. In some cases, a web application includes a media player element. In some cases, a media player element utilizes one or more of many suitable multimedia technologies including, by way of non-limiting examples, Adobe® Flash®, HTML 5, Apple® QuickTime®, Microsoft® Silverlight®, Java™, and Unity®.

In some cases, a computer program includes a mobile application provided to a mobile computing device. In some cases, the mobile application is provided to a mobile computing device at the time it is manufactured. In other cases, the mobile application is provided to a mobile computing device via the computer network described herein.

In view of the disclosure provided herein, a mobile application may be created using hardware, languages, and development environments known to the art. In some cases, mobile applications are written in several languages. Suitable programming languages may include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and PhoneGap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Several commercial forums may be available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, and Samsung® Apps.

In some cases, a computer program includes a standalone application, which is a program that is run as an independent computer process, not an add-on to an existing process, e.g., not a plug-in. Standalone applications may be compiled. A compiler may be a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some cases, a computer program includes one or more executable complied applications.

In some cases, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Web browser plug-ins may include Adobe® Flash® Player, Microsoft® Silverlight®, and Apple® QuickTime®. In some cases, the toolbar comprises one or more web browser extensions, add-ins, or add-ons. In some cases, the toolbar comprises one or more explorer bars, tool bands, or desk bands.

Several plug-in frameworks may be available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some cases, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

In some cases, the platforms, systems, media, and methods disclosed herein include software, server, or database modules, or use of the same. Software modules may be created by techniques using machines, software, and languages. The software modules disclosed herein are implemented in a multitude of ways. In some cases, a software module comprises a file, a section of code, a programming object, a programming structure, a distributed computing resource, a cloud computing resource, or combinations thereof. In some cases, a software module comprises a plurality of files, a plurality of sections of code, a plurality of programming objects, a plurality of programming structures, a plurality of distributed computing resources, a plurality of cloud computing resources, or combinations thereof. In some cases, the one or more software modules comprise, by way of non-limiting examples, a web application, a mobile application, a standalone application, and a distributed or cloud computing application. In some cases, software modules are in one computer program or application. In some cases, software modules are in more than one computer program or application. In some cases, software modules are hosted on one machine. In some cases, software modules are hosted on more than one machine. In some cases, software modules are hosted on a distributed computing platform such as a cloud computing platform. In some cases, software modules are hosted on one or more machines in one location. In some cases, software modules are hosted on one or more machines in more than one location.

In some cases, the platforms, systems, media, and methods disclosed herein include one or more databases, or use of the same. In some cases, various databases may be suitable for storage and retrieval of one or more of (i) wearable data, (ii) responses to health queries, (iii) geographic data, etc., one or more of which may be historical, present, or future data or information. In some cases, suitable databases include, by way of non-limiting examples, relational databases, non-relational databases, object oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some cases, a database is Internet-based. In further cases, a database is web-based. In still further cases, a database is cloud computing-based. In a particular case, a database is a distributed database. In other cases, a database is based on one or more local computer storage devices.

### Particular Implementations

In some cases, assembling a pool of high-risk subjects for developing an acute illness may include obtaining, from a plurality of subjects, (i) one or more responses to one or more health queries, and (ii) geographic incidence data for the acute illness. In some cases, a risk of developing the acute illness for the plurality of subjects may be predicted, using a machine learning model, based on the one or more responses and the geographic incidence data. In some cases, the pool of high-risk subjects may be identified from the plurality of subjects, where the risk of developing the acute illness for each subject of the pool of high-risk subjects satisfies a threshold. In some cases, the pool of high-risk subjects may be output.

In some cases, presenting information to a subject about an acute illness may include obtaining wearable device sensor data from the subject. In some cases a response to a health query of whether the subject has a symptom associated with the acute illness may be obtained via a mobile device. In some cases, a risk of the subject developing the acute illness may be predicted, using a machine learning model, based at least in part on the wearable device sensor data and the response to the health query. In some cases, an electronic display may be caused to indicate the risk to the subject.

### Additional Considerations

While various embodiments of the invention have been shown and described herein, it will be obvious to those skilled in the art that such embodiments are provided by way of example. Numerous variations, changes, and substitutions may occur to those skilled in the art without departing from the invention. It should be understood that various alternatives to the embodiments of the invention described herein may be employed.

It should be noted that various illustrative or suggested ranges set forth herein are specific to their example embodiments and are not intended to limit the scope or range of disclosed technologies, but, again, merely provide example ranges for frequency, amplitudes, etc. associated with their respective embodiments or use cases. Certain inventive embodiments herein contemplate numerical ranges. When ranges are present, the ranges include the range endpoints. Additionally, every sub range and value within the range is present as if explicitly written out.

While preferred embodiments of the present invention have been shown and described herein, it will be obvious to those skilled in the art that such embodiments are provided by way of example. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A computer-implemented method for assembling a pool of high-risk subjects for developing an acute illness, comprising:
obtaining, from a plurality of subjects, (i) one or more responses to one or more health queries, and (ii) geographic incidence data for the acute illness;
predicting, using a machine learning model, a risk of developing the acute illness for the plurality of subjects based on the one or more responses and the geographic incidence data;
identifying the pool of high-risk subjects from the plurality of subjects, wherein the risk of developing the acute illness for each subject of the pool of high-risk subjects satisfies a threshold; and
outputting the pool of high-risk subjects.

2. The computer-implemented method of claim 1, further comprising:
predicting, using a second machine learning model, an incidence rate for the acute illness for a population that comprises at least a portion of the plurality of subjects, wherein the second machine learning model processes data from the pool of high-risk subjects; and
displaying the incidence rate for the acute illness over the population.

3. The computer-implemented method of claim 2, wherein the data is wearable device sensor data.

4. The computer-implemented method of claim 2, wherein the incidence rate is predicted for a future time period.

5. The computer-implemented method of claim 1, wherein the one or more responses to the one or more health queries comprise physiological data that includes one or more of resting heart rate data, sleep data, step count data, blood pressure data, caloric data, nutrition data, or body temperature data.

6. The computer-implemented method of claim 1, wherein the acute illness is an infectious disease.

7. The computer-implemented method of claim 6, wherein the infectious disease is COVID-19 or a flu.

8. The computer-implemented method of claim 1, wherein the one or more responses to the one or more health queries comprise one or both of audio data or video data.

9. The computer-implemented method of claim 1, wherein the machine learning model comprises a decision tree algorithm.

10. The computer-implemented method of claim 9, wherein the decision tree algorithm comprises a random forest model.

11. The computer-implemented method of claim 1, wherein the machine learning model comprises a generative additive model.

12. The computer-implemented method of claim 11, wherein the geographic incidence data is state-wide data.

13. The computer-implemented method of claim 11, wherein the geographic incidence data is county-wide data.

14. The computer-implemented method of claim 11, wherein the geographic incidence data is associated with a plurality of zip code tabulation areas (ZCTAs).

15. The computer-implemented method of claim 1, wherein the one or more health queries comprise one or more of a household composition query, an occupation query, a residence query, or an infected contact query.

16. A system for presenting information to a subject about an acute illness, comprising:
one or more processors; and
one or more memories storing computer-executable instructions that, when executed, cause the one or more processors to:
(a) obtain wearable device sensor data from the subject;
(b) obtain, via a mobile device, a response to a health query of whether the subject has a symptom associated with the acute illness;
(c) predict, using a machine learning model, a risk of the subject developing the acute illness based at least in part on the wearable device sensor data and the response to the health query; and
(d) cause an electronic display to indicate the risk to the subject.

17. The system of claim 16, wherein the mobile device is a wearable device that collects the wearable device sensor data from the subject.

18. The system of claim 17, wherein the wearable device comprises the electronic display.

19. The system of claim 18, wherein the computer-executable instructions, when executed, further cause the one or more processors to:
cause the electronic display of the wearable device to present the wearable device sensor data to the subject.

20. The system of claim 16, wherein the computer-executable instructions, when executed, further cause the one or more processors to:
determine the risk of the subject developing the acute illness satisfies a threshold; and
in response to determining the risk of the subject developing the acute illness satisfies the threshold, request, via the mobile device, a response to a health query of whether the subject has one or more additional symptoms of the acute illness.

21. The system of claim 16, wherein the computer-executable instructions, when executed, further cause the one or more processors to:
obtain, via the mobile device, demographic data of the subject.

22. The system of claim 21, wherein the demographic data comprises one or more of race data, ethnicity data, gender data, education data, or age data.

23. The system of claim 16, wherein the computer-executable instructions, when executed, further cause the one or more processors to perform operations (a) and (b) on a repeating basis at a first time interval.

24. The system of claim 23, wherein the computer-executable instructions, when executed, further cause the one or more processors to perform operations (c) and (d) on a repeating basis at a second time interval that is longer than the first time interval.
